(19) United States
(12) Patent Application Publication
(10) Pub. No.: US 2002/0128232 A1
(43) Pub. Date: Sep. 12, 2002

(54) Heterocyclic angiogenesis inhibitors

(76) Inventors: Scott A. Henderson, Rowville (AU); Barry R. Matthews, Olinda (AU)

Correspondence Address: Stephen A. Bent, FOLEY & LARDNER, Washington Harbour, 3000 K Street, N.W., Suite 500, Washington, DC, 20007-5109, US

(21) Appl. No.: 09/975,315

(22) Filed: Oct. 12, 2001

Related U.S. Application Data
(60) Provisional application No. 60/239,276, filed on Oct. 12, 2000.

(51) Int. Cl. A61K 31/69; A61K 31/675; A61K 31/433; A61K 31/4184

(52) U.S. Cl. 514/79; 514/362; 514/394; 514/64; 548/112; 548/110; 548/134; 548/306.4; 548/307.1; 548/307.4

(57) ABSTRACT

The present invention relates to novel angiogenic-inhibitory compounds of formula (I)

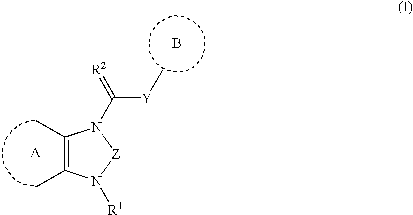

and pharmaceutically acceptable salts thereof, wherein:

Y is a direct bond or a linker group selected from a group of CH₂, NH, NR₁, S, SO, SO₂, or O;

Z is CO, CS, SO, SO₂, or C═NH;

R¹ is H, alkyl, alkenyl, alkynyl, cycloalkyl, aryl, arylalkyl, acyl, alkoxyacyl, aryloxyacyl, or aminoacyl groups;

R² is O, S, or NH;

A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy and n is 0-11; and

B is alkyl, arylalkyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy and n is 0-11, pharmaceutical composition comprising them and their use.

This application claims priority based on U.S. Provisional Application No. 60/239,276, filed on Oct. 12, 2000.

### TECHNICAL FIELD

The present invention relates to novel compounds capable of modulating, regulating and/or inhibiting angiogenesis. The present invention is also directed to methods of modulating, regulating and/or inhibiting angiogenesis, preferably, for prevention and/therapeutic treatment of disorders related to unregulated angiogenesis, including cell proliferative disorders.

### BACKGROUND OF THE INVENTION

Angiogenesis is the formation of new capillary blood vessels from existing ones. It is a multi-step process, tightly controlled by the balance between angiogenic promoters and inhibitors. Pathological processes such as atherosclerosis, diabetic retinopathy, psoriasis, rheumatoid arthritis, and tumor growth result from an imbalance in angiogenesis factors. In healthy adult tissue angiogenesis occurs only in pregnancy, wound healing, and corpus luteum formation. Although angiogenesis is a highly regulated process under normal conditions, many diseases involving angiogenesis are driven by persistent unregulated angiogenesis. Otherwise stated, unregulated angiogenesis may either cause a particular disease directly or exacerbate an existing pathological condition.

The concept of developing compounds to inhibit angiogenesis offers hope of new treatments for these diseases involving angiogenesis. Folkman, J. New Engl. J. Med. 285: 1182-1186, 1971; Klagsbrun, M. et al., Chem & Biol. 6: R217-R224, 1999. For example, by targeting the endothelial cells associated with tumors such drugs would have a number of potential advantages. Those advantages include low resistance, as endothelial cells are genomically more stable than tumor cells; low toxicity, since endothelial cells are normally quiescent, with extremely long turnovers; and the possibility for use in combination with cytotoxic drugs. For these reasons, research into angiogenesis inhibitors has focused on cancer therapy. Gasparini, G. Drugs 58(1): 17-38; 1999; Brehm, S. Angiogenesis 2: 9-20, 1998; Giannis, A. et al., Angew. Chem. Int. Ed. Engl. 36: 588-590, 1997; Risau, W. Nature 386: 671-674, 1997.

It has been shown experimentally in animals that solid tumor growth is dependent on angiogenesis. Folkman, J. New Engl. J. Med. 285: 1182-1186, 1971. Tumor metastasis is also dependent on angiogenesis. Folkman, J. Nat. Med. 1: 27-31, 1995; Folkman, J. New Engl. J. Med. 333: 1757-1763, 1995. Folkman's group has demonstrated that repeated courses of treatment with angiogenesis inhibitors do not induce tumor resistance. Boehm, T. et al., Nature 390: 404-407, 1997. Angiogenesis inhibitors may also be useful for treatment of other angiogenesis-dependent diseases such as diabetic retinopathy, (Hammes, H. P. et al., Nat. Med. 2: 529-533, 1996), rheumatoid arthritis, (Storgard, C. M. et al. J. Clin. Invest. 103: 47-54, 1999), Kaposi's sarcoma, and von Hipple-Lindau disease.

Several angiogenesis inhibitors are under development, for use in treating diseases involving angiogenesis, (Gasparini, G. et al., J Clin. Oncol. 13(3): 765-782, 1995), but there are disadvantages involved. For example, suramin is a potent angiogenesis inhibitor but causes, at doses required to reach antitumor activity, severe systemic toxicity in humans. Other compounds, such as retinoids, interferons and antiestrogens, are safe for human use but have only a weak anti-angiogenic effect. Still other compounds may be difficult or costly to make. For example, angiostatin may be generated by elastase digestion of lys-plasminogen, but preferentially generates a polypeptide containing kringles 1-3. This necessitates the use of large amounts of starting material in order to obtain sufficient quantities of kringle 1-4 polypeptide (i.e., angiostatin). As a result, a number of angiogenesis inhibitors are currently in clinical trials, but as yet none is commercially available. Klohs, W. D., et al., Curr. Op. Biotechnol. 10: 544-549, 1999; Hamby, J. M. et al. Pharmacol. Ther. 82: 169-193, 1999; Boschelli, D. H. Drugs of the Future 24(5): 515-537, 1999.

Thus, there is a need for compounds for treating a variety of diseases involving angiogenesis. More specifically, there is a need for angiogenesis inhibitors that are safe for therapeutic use and that exhibit selective toxicity with respect to the pathological condition. Furthermore, such compounds should be readily produced and cost-effective.

### SUMMARY OF THE INVENTION

The present inventors have discovered a class of organic molecules that are capable of modulating/regulating angiogenesis. Without limiting the invention to a particular mechanism, the inventors believe that the molecules act by tyrosine kinase inhibition. In any event, the compounds of the present invention are useful for treating diseases in which angiogenesis plays a role; illustrative of these are diabetic retinopathy, atherosclerosis, psoriasis, and rheumatoid arthritis, as well as the more general processes of both tumor metastasis and growth. The present invention also allows for the identification of compounds that specifically inhibit angiogenesis, in order to regulate and/or modulate abnormal or inappropriate cell proliferation.

Accordingly, it is an object of the present invention to provide angiogenic-inhibitory compounds having the following formula:

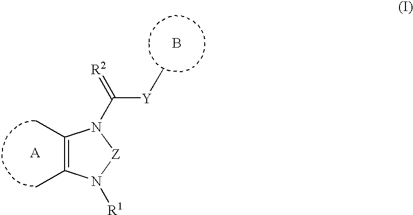

and pharmaceutically acceptable salts thereof, wherein:

Y is a direct bond or a linker group selected from a group of CH₂, NH, NR₁, S, SO, SO₂, or O;

Z is CO, CS, SO, SO₂, or C═NH;

R¹ is H, alkyl, alkenyl, alkynyl, cycloalkyl, aryl, arylalkyl, acyl, alkoxyacyl, aryloxyacyl, or aminoacyl groups;

R² is O, S, or NH;

A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; and

B is alkyl, arylalkyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy; and n is 0-11, excluding the following compounds:

(i) a compound, wherein Y is a direct bond, Z is CO, R¹ is 1-(piperidin-4-yl)piperidin-4-yl, R² is O, A is phenyl, and B is methyl;

(ii) compounds, wherein Y is NH, Z is CO, R¹ is H, R² is O, A is phenyl, and B is phenyl, bromophenyl, mono/dichlorophenyl, methoxyphenyl, tolyl, nitrophenyl, 3-trifluoromethylphenyl, benzoic acid, phenylsulfonylaminophenyl, 1-naphthalenyl, 4-piperidinyl, azabicyclooctyl, or azabicyclononyl;

(iii) compounds, wherein Y is NH, Z is CO, R¹ is H, R² is O, A is nitrophenyl, and B is phenyl, 4-bromophenyl, or 3,4-dichlorophenyl;

(iv) compounds, wherein Y is NH, Z is CO, R¹ is H, R² is O, A is mono/dichlorophenyl, and B is 4-bromophenyl, phenyl, dichlorophenyl, mono/di alkoxyphenyl, 1-piperidinyl, or isoindol-5-yl;

(v) compounds, wherein Y is NH, Z is CO, R¹ is H, R² is O, A is mono/dimethylphenyl, and B is phenyl or methylphenyl;

(vi) a compound, wherein Y is NH, Z is CO, R¹ is H, R² is O, A is naphthalenyl, and B is phenyl;

(vii) compounds, wherein Y is NH, Z is CO, R² is O, A is phenyl, R¹ is i-propyl, and B is 1-piperazinyl, 4-pyridinyl, 4-piperidinyl, or methanopyrolizin-1-yl;

(viii) compounds, wherein Y is NH, Z is CO, R² is O, A is phenyl, R¹ is alkyl, alkenyl, or alkynyl, and B is azabicyclooctyl, azabicyclononyl, or azabicyclodecyl;

(ix) a compound, wherein Y is NMe, Z is CO, R² is O, R¹ is H, A is phenyl, and B is azabicyclononyl;

(x) a compound, wherein Y is NPh, Z is CO, R² is O, R¹ is H, and A and B are phenyl;

(xi) a compound, wherein Y is NR, R is dimethoxyphenylmethyl, Z is CO, R² is O, R¹ is H, A is phenyl, and B is azabicyclononyl;

(xii) a compound, wherein Y is NPh, Z is CO, R² is O, B is phenyl, R¹ and A taken together to form guanosine derivatives; and

(xiii) compounds, wherein Y is a NH, Z is CS, R¹ is H, R² is S, A is phenyl, and B is aminophenyl, benzoic acid, benzyl, butyl, hydroxyphenyl, chlorophenyl, methyl, morpholinyl, propyl, phenyl, pyridinyl, or thiophene-2-carboxylate.

It is yet another object of the present invention to provide pharmaceutical compositions that comprise a pharmaceutically acceptable carrier or excipient and a therapeutically effective amount of a compound of the formula:

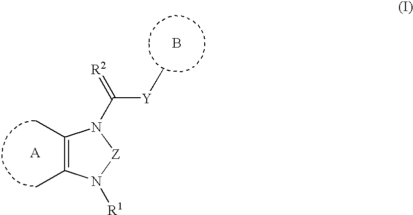

or pharmaceutically acceptable salts thereof, wherein:

Y is a direct bond or a linker group selected from a group of CH₂, NH, NR₁, S, SO, SO₂, or O;

Z is CO, CS, SO, SO₂, or C═NH;

R¹ is H, alkyl, alkenyl, alkynyl, cycloalkyl, aryl, arylalkyl, acyl, alkoxyacyl, aryloxyacyl, or aminoacyl groups;

R² is O, S, or NH;

A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy,and n is 0-11; and

B is alkyl, arylkalyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy; and n is 0-11, excluding the following compounds:

(i) a compound, wherein Y is a direct bond, Z is CO, R¹ is 1-(piperidin-4-yl)piperidin-4-yl, R² is O, A is phenyl, and B is methyl;

(ii) compounds, wherein Y is NH, Z is CO, R¹ is H, R² is O, A is phenyl, and B is N-substituted aminomethylphenyl, azabicyclooctyl, or azabicyclononyl;

(iii) compounds, wherein Y is NH, Z is CO, R² is O, A is phenyl, R¹ is alkyl, alkenyl, or alkynyl, and B is azabicyclooctyl, azabicyclononyl, or azabicyclodecyl; and

(iv) a compound, wherein Y is NMe, Z is CO, R² is O, R¹ is H, A is phenyl, and B is azabicyclononyl.

Such a composition is believed to modulate/regulate and/or inhibit angiogenesis; possibly, but not necessarily, by tyrosine kinase inhibition. Thus, it is another object of the present invention to provide a method for regulating, modulating, or inhibiting angiogenesis, comprising administrating to a subject a therapeutically effective amount of a compound of the formula:

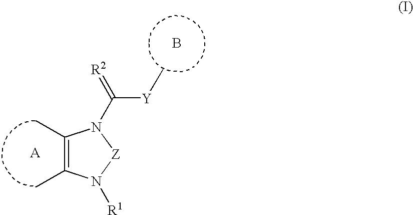

or pharmaceutically acceptable salts thereof, wherein:

Y is a direct bond or a linker group selected from a group of CH₂, NH, NR₁, S, SO, SO₂, or O;

Z is CO, CS, SO, SO₂, or C═NH;

R¹ is H, alkyl, alkenyl, alkynyl, cycloalkyl, aryl, arylalkyl, acyl, alkoxyacyl, aryloxyacyl, or aminoacyl groups;

R² is O, S, or NH;

A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; and

B is alkyl, arylalkyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy; and n is 0-11.

The compositions of the present invention may be used for preventive or therapeutic treatment of diseases involving angiogenesis, such as atherosclerosis, diabetic retinopathy, psoriasis, rheumatoid arthritis, and tumor growth. Therefore, it is still another object of the present invention to provide a method for preventive or therapeutic treatment of such diseases involving angiogenesis, by administering a therapeutically effective amount of the above described compounds of Formula I to a patient in need thereof.

The compounds of the present invention or pharmaceutical compositions containing compounds of the present invention may also be used in conjunction with other treatments for the prevention or therapy of diseases involving angiogenesis. For example in the case of treating tumour growth or metastases, the compounds or pharmaceutical compositions can be used in conjunction with radiation, radioimmunotherapy, chemotherapy, or in combination with other angiogenesis inhibitors.

### DETAILED DESCRIPTION OF PREFERRED EMBODIMENTS

In the context of this description, the term “alkyl” refers to an unsubstituted or substituted, straight-chain or branched saturated aliphatic hydrocarbon radical. Preferably the alkyl group has 1 to 12 carbons as exemplified by methyl, ethyl, propyl, isopropyl, butyl, isobutyl, sec-butyl, t-butyl, pentyl, i-pentyl, hexyl, heptyl, octyl and the like. The alkyl group is optionally substituted with one or more substituents selected from hydroxyl, cyano, alkoxy, ═O, ═S, NO₂, halogen, amino, or SH.

The term “alkenyl” refers to an unsubstituted or substituted, straight chain or branched hydrocarbon radical having 2 to about 12 carbon atoms, as exemplified by vinyl, propenyl, 2-butenyl, 3-butenyl, isobutenyl and 2-octenyl.

The term “alkynyl” refers to an unsubstituted or substituted, straight chain or branched hydrocarbon radical having 2 to about 12 carbon atoms, as exemplified by ethynyl, 2-propynyl, 2-butynyl, 2-pentynyl and 2-octynyl.

The term “cycloalkyl” refers to at least one three- to eight-member, non-heterocyclic (i.e., carbocyclic) or heterocyclic ring. Such a ring preferably has from one to three ring structures, where the ring has three to eight members, in a predominantly planar configuration. Exemplary of a non-heterocyclic ring in this regard is a substituted or unsubstituted cyclopropane, cyclobutane, cyclopentane, cycloheptane, cyclohexane, cyclohexanedione, cyclopentanedione, quinone, and tricyclododecane. Suitable heterocycloalkyl groups include substituted or unsubstituted azetidinyl, pyrrolidinyl, piperidinyl, piperazinyl and morpholinyl groups. The cycloalkyl group is optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′ wherein R and R′ are each independently selected from the group consisting of H, alkyl, alkoxy, aryl, aryloxy, arylalkyl, and arylalkyloxy; and n is 0-11.

The term “aryl” refers to an aromatic group, constituted by a cycle or condensed cycles; each cycle can optionally contain one or more identical or different heteroatoms, chosen from sulphur, nitrogen or oxygen. Examples of an aryl group are pyrrolidine, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene. The aryl group is optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy; and n is 0-11.

The term “alkoxy” refers to O-alkyl groups wherein “alkyl” is as defined as above. The methoxy, ethoxy, propoxy, isopropyloxy or tert-butyloxy radicals are preferred.

The term “arylalkyl” refers to an aryl-substituted alkyl group wherein “aryl” and “alkyl” are as defined as above. Preferred arylalkyl groups include, for example, benzyl, diphenylmethyl, triphenylmethyl, diphenylethyl phenylethyl, phenylbutyl and phenylpropyl. Such groups may be substituted or unsubstituted.

The term “acyl” refers a radical which could be formed by removal of the hydroxy from a carboxylic acid (i.e., R—C[═O]—). Preferred acyl groups include, for example, acetyl, formyl, and propionyl.

The term “alkoxyacyl” is an acyl radical (—C[═O]—) having an alkoxy substituent (i.e., —O—R), for example, —C(═O)—O-alkyl. Such groups may be substituted or unsubstituted.

The term “aryloxy” refers to O-aryl groups wherein “aryl” is as defined as above. Such groups may be substituted or unsubstituted.

The term “aryloxyacyl” refers to acyl groups wherein “aryloxy” is as defined as above. Such groups may be substituted or unsubstituted.

The term “aminoacyl” refers to acyl groups having an amino substituent (i.e., —C(═O)—N); for example, —C(═O)—NH₂. The amino group of the aminoacyl moiety may be unsubstituted (i.e., primary amine) or may be substituted with one (secondary amine) or two (i.e., tertiary amine) alkyl groups.

The term “arylalkyloxy” refers to O-arylalkyl groups wherein “aryl” and “alkyl” are as defined as above. Such groups may be substituted or unsubstituted.

Preferred groups represented by A and B are cycloalkyl or aryl ring groups, present as a single ring, or condensed with other ring, wherein each ring is selected from the group consisting of cyclopropane, cyclobutane, cyclopentane, cycloheptane, cyclohexane, cyclohexanedione, cyclopentanedione, quinone, tricyclododecane, pyrrolidine, piperidine, piperazine, morpholine, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene, optionally substituted at one or more positions with alkyl, alkoxy, aryl, aryloxy, alkaryl, alkaryloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy; and n is 0-11.

“Pharmaceutically acceptable salt” refers to a formulation of a compound that is non-toxic and does not abrogate the biological activity and properties of the compound. Said salts can conveniently be obtained by treating either the basic forms of the compounds of Formula (I) with appropriate organic or inorganic acids, or by treating the acidic forms of the compounds of Formula (I) with appropriate organic or inorganic bases. Examples of the inorganic acids which may be employed to form pharmaceutically acceptable salts include such inorganic acids as hydrochloric, hydrobromic, hydroiodic, nitric, carbonic, sulfuric and phosphoric acid. Suitable pharmaceutically acceptable acid addition salts include but are not limited to the following: acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxy-ethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Water or oil-soluble or dispersible products are thereby obtained. Suitable, pharmaceutically acceptable base addition salts include, for example, metallic salts made from aluminium, calcium, lithium, magnesium, potassium, sodium and zinc, and organic salts made from N,N′-dibenzylethylenediamine, chloroprocaine, choline, diethanolamine, ethylenediamine, meglumine (N-methylglucamine) and procaine.

The compounds encompassed by the foregoing chemical formulae may exhibit tautomerism or structural isomerism. Thus, while any given formula depicts one possible tautomeric or structural isomeric form, it should be understood that the invention encompasses any tautomeric or structural isomeric form, or mixtures thereof, possessing the ability to regulate, modulate and/or inhibit angiogenesis and is not limited to any one tautomeric or structural isomeric form utilised within the formulae drawing.

The invention is further directed to solvated and unsolvated forms of the compounds, and their pharmaceutically acceptable salts, having the ability to regulate, modulate and/or inhibit angiogenesis.

In one illustrative embodiment, the invention provides compounds having the formula:

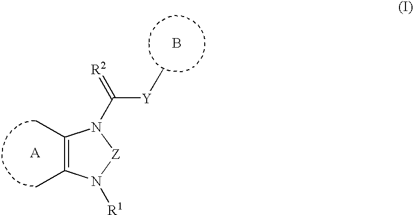

and pharmaceutically acceptable salts thereof, wherein:

Y is CH₂, NH, NR₁, S, SO, SO₂, or O;

R¹ is H, alkyl, alkenyl, alkynyl, cycloalkyl, aryl, arylalkyl, acyl, alkoxyacyl, aryloxyacyl, or aminoacyl groups;

R² is O, S, or NH each selected independently;

A is cycloalkyl or aryl ring groups, present as a single ring or condensed with other ring, wherein each ring is selected from cyclopropane, cyclobutane, cyclopentane, cycloheptane, cyclohexane, cyclohexanedione, cyclopentanedione, quinone, tricyclododecane, pyrrolidine, piperidine, piperazine, morpholine, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene, optionally substituted at one or more positions with alkyl, alkoxy, aryl, aryloxy, alkaryl, alkaryloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; and

B is alkyl, arylalkyl, or cycloalkyl or aryl ring groups present as a single ring or condensed with other ring, wherein each ring is selected from cyclopropane, cyclobutane, cyclopentane, cycloheptane, cyclohexane, cyclohexanedione, cyclopentanedione, quinone, tricyclododecane, pyrrolidine, piperidine, piperazine, morpholine, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene, optionally substituted at one or more positions with alkyl, alkoxy, aryl, aryloxy, alkaryl, alkaryloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11.

In another embodiment, the compounds of the present invention have the formula:

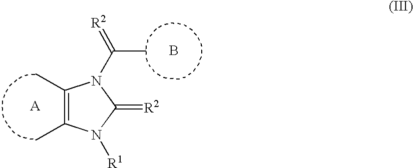

and pharmaceutically acceptable salts thereof, wherein:

R¹ is H, alkyl, alkenyl, alkynyl, cycloalkyl, aryl, arylalkyl, acyl, alkoxyacyl, aryloxyacyl, or aminoacyl groups;

R² is O, S, or NH each selected independently; and

A and B are as defined above.

In another embodiment, the compounds of the present invention have the formula:

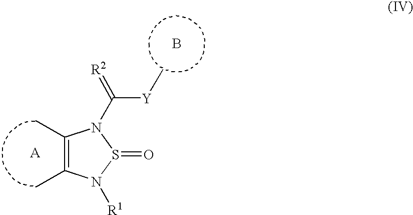

and pharmaceutically acceptable salts thereof, wherein:

Y is CH₂, NH, NR₁, S, SO, SO₂, or O;

R¹ is H, alkyl, alkenyl, alkynyl, cycloalkyl, aryl, arylalkyl, acyl, alkoxyacyl, aryloxyacyl, or aminoacyl groups;

R² is O, S, or NH; and

A and B are as defined above.

In another illustrative embodiment, the compounds of the present invention have the formula:

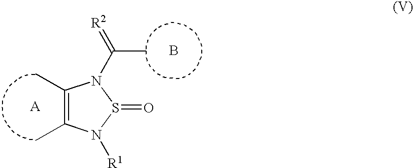

and pharmaceutically acceptable salts thereof, wherein:

R¹ is H, alkyl, alkenyl, alkynyl, cycloalkyl, aryl, arylalkyl, acyl, alkoxyacyl, aryloxyacyl, or aminoacyl groups;

R² is O, S, or NH; and

A and B are as defined above.

In another embodiment, the compounds of the present invention have the formula:

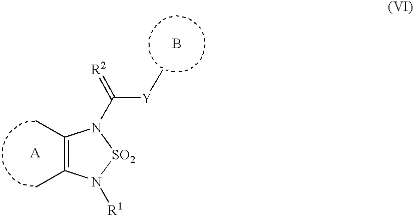

and pharmaceutically acceptable salts thereof, wherein:

Y is CH₂, NH, NR₁, S, SO, SO₂, or O;

R¹ is H, alkyl, alkenyl, alkynyl, cycloalkyl, aryl, arylalkyl, acyl, alkoxyacyl, aryloxyacyl, or aminoacyl groups;

R² is O, S, or NH; and

A and B are as defined above.

In another embodiment, the compounds of the present invention have the formula:

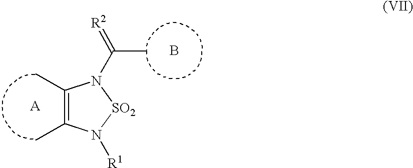

and pharmaceutically acceptable salts thereof, wherein:

R¹ is H, alkyl, alkenyl, alkynyl, cycloalkyl, aryl, arylalkyl, acyl, alkoxyacyl, aryloxyacyl, or aminoacyl groups;

R² is O, S, or NH; and

A and B are as defined above.

Preferred compounds include a compound of formula I wherein Y is direct bond or NH; Z is CO, CS or SO₂; R¹ is H, alkyl, cycloalkyl, aryl or arylalkyl; R² is O or S, A is substituted or unsubstituted phenyl, pyridinyl or pyrimidinyl; and B is substituted or unsubstituted aryl, alkyl, alkenyl, arylalkyl or cycloalkyl.

More preferred compounds include:

N-(4-Nitrophenyl)-2,3-dihydro-2-oxo-1H-benzimidazole-1-carboxamide;

N-(Phenyl-3-boronic acid)-2,3-dihydro-2-oxo-1H-benzimidazole-1-carboxamide;

N-(3-Carboxyphenyl)-2,3-dihydro-2-oxo-1H-benzimidazole-1-carboxamide;

N-(3-Carboxyphenyl)-2,3-dihydro-2-oxo-1H-benzimidazole-1-carboxamide Sodium salt;

N-(4-Carboxyphenyl)-2,3-dihydro-2-oxo-1H-benzimidazole-1-carboxamide;

N-(2-Carboxyphenyl)-2,3-dihydro-2-oxo-1H-benzimidazole-1-carboxamide;

N-(3-Carboxymethylphenyl)-2,3-dihydro-2-oxo-1H-benzimidazole-1-carboxamide;

N-(4-Carboxymethylphenyl)-2,3-dihydro-2-oxo-1H-benzimidazole-1-carboxamide;

N-(4-Ethoxycarbonylphenyl)-2,3-dihydro-2-oxo-1H-benzimidazole-1-carboxamide;

N-(3-Ethoxycarbonylphenyl)-2,3-dihydro-2-oxo-1H-benzimidazole-1-carboxamide;

2-(2,3-Dihydro-2-oxo-benzimidazole-1-carbonyl)-pyrrolidine-1-carboxyamide;

2-thioxo-2,3-dihydrobenzoimidazole-1-carboxylic acid phenylamide;

1-(pyrrolidine-2-carbonyl)-1,3-dihydrobenzoimidazol-2-one trifluoroacetic acid salt;

2-oxo-2,3-dihydrobenzoimidazole-1-carboxylic acid allylamide;

8-oxo-7,8-dihydropurine-9-carboxylic acid phenylamide;

2-oxo-2,3-dihydrobenzoimidazole-1-carboxylic acid t-butylamide;

2-oxo-2,3-dihydrobenzoimidazole-1-carboxylic acid (4-t-butylphenyl)amide;

2-oxo-2,3-dihydrobenzimidazole-1-carboxylic acid phenylamide;

2-oxo-2,3-dihydroimidazole[4,5-c]pyridine-1-carboxylic acid m-tolylamide;

2,2-dioxo-2,3-dihydro-2λ⁶-benzo[1,2,5]thiadiazole-1-carboxylic acid

(3,5-dimethoxyphenyl)amide; 2-oxo-2,3-dihydrobenzimidazole-1-carboxylic acid

(1,1,3-trioxo-2,3-dihydro-1H 1λ⁶-benzo[d]isothiazol-5-yl)amide;

2-hydroxy-5-[(2-oxo-2,3-dihydrobenzimidazole-1-carbonyl)-amino]benzoic acid;

4-{[(2-oxo-2,3-dihydrobenzimidazole-1-carbonyl)-amino]-methyl}benzoic acid;

5-[(2-oxo-2,3-dihydrobenzimidazole-1-carbonyl)-amino]isophthalic acid;

2-oxo-2,3-dihydrobenzimidazole-1-carboxylic acid (4-sulfamoylphenyl)amide;

2-oxo-2,3-dihydrobenzimidazole-1-carboxylic acid (3-chlorophenyl)amide; phosphoric acid

mono-{4[(2-oxo-2,3-dihydrobenzimidazole-1-carbonyl)-amino]-phenyl} ester;

2-oxo-2,3-dihydrobenzimidazole-1-carboxylic acid biphenyl-4-ylamide;

N-(phenyl-4-boronic acid)-2,3-dihydro-2-oxo-1H-benzimidazole-1-carboxamide; 2-oxo-2,3-dihydrobenzimidazole-1-carbothioic acid (3-trifluoromethylphenyl)amide; 2-oxo-2,3-dihydrobenzimidazole-1-carbothioic acid p-tolylamide; 2-thioxo-2,3-dihydrobenzimidazole-1-carbothioic acid (3-trifluoromethylphenyl) amide; 2-thioxo-2,3-dihydrobenzimidazole-1-carbothioic acid p-tolylamide; 2-oxo-2,3-dihydrobenzimidazole-1-carboxylic acid cyclohexylamide; 5-methoxy-2-oxo-2,3-dihydrobenzimidazole-1-carboxylic acid phenylamide; and 2-oxo-2,3-dihydrobenzimidazole-1-carboxylic acid (4,6-dimethylpyrimidin-2-yl) amide.

The present invention relates to compounds capable of modulating/regulating and/or inhibiting angiogenesis, possibly but not necessarily by tyrosine kinase inhibition, for preventive and/or therapeutic treatment of proliferative, fibrotic, or metabolic diseases. This maladies include, for example, atherosclerosis, arthritis, cancer, diabetic retinopathy, fibrosis, psoriasis, and restenosis.

More particularly, the present invention is directed to compounds that modulate/regulate and/or inhibit angiogenesis for preventive and/or therapeutic treatment of cancer, including astrocytoma, carcinoma, erythroblastoma, glioblastoma, leukemia, melanoma, meningioma, myoblastoma, and sarcoma. Indications may include, but are not limited to bladder cancers, blood cancers, bone cancers, brain cancers, colon cancers, gastric cancers, lung cancers, ovarian cancers, and pancreas cancers.

In view of the usefulness of the subject compounds in the preventive or therapeutic treatment of angiogenesis dependent disorders, the present invention provides a method of treating mammals suffering from such disorders, said method comprising the systemic administration of a therapeutic effective amount of a compound of Formula (I), or a pharmaceutically acceptable salt thereof.

The compounds of the present invention or pharmaceutical compositions containing these compounds may also be used in conjunction with other treatments for the prevention or therapy of diseases involving angiogenesis. For example in the case of treating tumour growth or metastases, the compounds or pharmaceutical compositions can be used in conjunction with radiation, radioimmunotherapy, chemotherapy, or in combination with other angiogenesis inhibitors.

In view of their useful pharmacological properties, the subject compounds may be formulated into various pharmaceutical forms for administration purposes. To prepare the pharmaceutical compositions of this invention, an effective amount of a particular compound, in base or acid addition salt form, as the active ingredient is combined in intimate admixture with a pharmaceutically acceptable carrier, which carrier may take a wide variety of forms depending on the form of preparation desired for administration. These pharmaceutical compositions are desirably in unitary dosage form suitable, preferably, for administration orally, rectally, percutaneously, or by parenterally. Alternatively, a compound of the present invention may be administered as pharmaceutical compositions containing the compound of interest in combination with one or more pharmaceutically acceptable excipients. A pharmaceutically acceptable carrier or excipient refers to a non-toxic solid, semi-solid or liquid filler, diluent, encapsulating material or formulation auxiliary of any type. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelation and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral compositions, the carrier will usually comprise sterile water, at least in large part, though other ingredients, to aid solubility for example, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect to the skin. Said additives may facilitate the administration to the skin and/or may be helpful for preparing the desired compositions. These compositions may be administered in various ways, e.g., as a transdermal patch, as a spot-on, as an ointment. It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

When used in the above or other treatments, a therapeutically effective amount of one of the compounds of the present invention may be employed in pure form or, where such forms exist, in pharmaceutically acceptable salt form. By a “therapeutically effective amount” of the compound of the invention is meant a sufficient amount of the compound to treat an angiogenic disease, (for example, to limit tumor growth or to slow or block tumor metastasis) at a reasonable benefit/risk ratio applicable to any preventive or therapeutic medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved.

In light of the present commentary and the results detailed herein, a person familiar with the pharmaceutical testing will understand the routine nature of determining an effective amount of a given compound with the invention. Thus, determining a therapeutically effective amount is well within the purview of the skilled clinician, and will depend on the exact identity of the inventive compound and particular patient characteristics, inter alia. General guidance can be found, for example, in the publications of the International Conference on Harmonisation.

Such a determination specifically will depend on such factors as the toxicity and efficacy profile of a given, inventive compound. In an initial clinical trial, a patient in need of treatment or a normal volunteer typically is administered an inventive compound at a specified dose, usually low, at, specified intervals for a period of time. In the absence of adverse effects, as determined by the clinician, this procedure may be repeated with successively higher doses of inventive compound. In this way, potential toxic side-effects and parameters, such as bioavailability, may be determined using methods readily known in the art. Some typical pre-clinical and clinical parameters that are monitored are found in Remington's Pharmaceutical Sciences, chapters 27-28, pages 484-528 (Mack Publishing Company, 1990). With the results of the toxicology studies in mind, clinical trials for efficacy are undertaken.

In general, it is contemplated that an effective amount would be from 10⁻⁵ mg/kg to 100 mg/kg body weight, and in particular from 0.001 mg/kg to 10 mg/kg body weight. It may be appropriate to administer the required dose as two, three, four or more sub-doses, at appropriate intervals throughout the day. The sub-doses may be formulated as unit dosage forms, for example, containing between about 0.001 to 500 mg, and particularly 0.01 mg to 200 mg of active ingredient per unit dosage form.

The compounds of the present invention may be synthesised by known techniques. The following examples are included by way of illustration, not limitation of the invention.

### EXAMPLE 1

### N-(4-Nitrophenyl)-2 3-dihydro-2-oxo-1H-benzimidazole-1-carboxamide

A suspension of 4-nitrobenzylisocyanate (1.14 g, 6.9 mmol) in DMF (5 ml) was added dropwise to a solution of 2-hydroxybenzimidazole (1.03 g, 7.7 mmol) in DMF (10 ml). A precipitate formed almost immediately. The mixture was stirred under a nitrogen atmosphere, at room temperature for 15 h. The precipitate was collected by filtration, washed with DMF followed by diethyl ether. The crude product contained a mixture of 4-nitroaniline, 2-hydroxybenzimidazole, bis-nitrophenylurea, and desired product. The filtrate also contained desired product and was concentrated in vacuo. Trituration with ethanol, acetone (50° C.), and ethanol again gave N-(4-nitrophenyl)-2,3-dihydro-2-oxo-1H-benzimidazole-1-carboxamide (0.46 g, 22%). ¹H NMR (200 MHz, d₆-DMSO) δ7.05-7.30 (m, 3H), 7.87 (d, J=5.0 Hz, 2H), 8.01 (d, J=6 Hz, 1H), 8.27 (d, J=5.0 Hz, 2H), 11.40 (s, 1H), 11.80 (br s, 1H); ¹³C NMR (50 MHz, d₆-DMSO) δ109.3, 114.4, 119.3, 122.0, 124.1, 125.0, 126.7, 127.9, 142.6, 143.4, 148.5, 153.5; MS (APCI−) m/z 297 (M−H).

### EXAMPLE 2 0

### N-(Phenyl-3-boronic acid)-2,3-dihydro-2-oxo-1H-benzimidazole-1-carboxamide

A solution of triphosgene (1.67 g) in THF (5 ml) was added to a stirred solution of 2-hydroxybenzimidazole (1.01 g) and activated carbon (0.03 g) in THF (20 ml). Mixture heated at reflux for 8 h and stirred another 10 h at room temperature. The solution was filtered and the filtrate concentrated in vacuo. The crude product was triturated with diethyl ether, collected by filtration, and dried in vacuo to give 2-hydroxybenzimidazolyl chloroformate as a yellow solid (0.94 g, 85%). The product was used directly for the next step as per the literature. Tapia, I., et al., J. Med. Chem. 42: 2870-2880, 1999.

2-Hydroxybenzimidazolyl chloroformate (1.88 g, 9.6 mmol) was dissolved in THF (50 ml) and 3-aminophenylboronic acid (1.56 g, 10.1 mmol) added. Mixture was stirred at room temperature for 15 h. The precipitate was collected by filtration and washed with water (30 ml) and ethanol (2×30 ml). The product was dried in vacuo to give N-(phenyl-3-boronic acid)-2,3-dihydro-2-oxo-1H-benzimidazole-1-carboxamide as a pale yellow solid (1.43 g, 50% yield). ¹H NMR (200 MHz, d₆-DMSO) δ7.05-7.25 (m, 3H), 7.36 (t, J=7.7 Hz, 1H), 7.56 (d, J=7.3 Hz, 1H), 7.79 (d, J=8.7 Hz, 1H), 7.81 (s, 1H), 8.04 (d, J=7.7 Hz, 1H), 8.10 (br s, 1H), 10.92 (s, 1H), 11.88 (s, 1H); ¹³C NMR (50 MHz, d₆-DMSO) δ109.7, 114.5, 121.5, 122.0, 123.9, 125.5, 127.2, 127.9, 128.3, 129.9, 135.3, 136.5, 148.8, 153.8; MS (ES−) m/z 296 (M−H).

### EXAMPLE 3

### N-(3-Carboxyphenyl)-2,3-dihydro-2-oxo-1H-benzimidazole-1-carboxamide

2-Hydroxybenzimidazolyl chloroformate (0.17 g, 0.8 mmol) was dissolved in THF (2 ml) and 3-aminobenzoic acid (0.12 g, 0.9 mmol) added. Mixture was stirred at room temperature for 15 h. The mixture was concentrated under reduced pressure and the residue triturated with water and ethanol. N-(3-Carboxyphenyl)-2,3-dihydro-2-oxo-1H-benzimidazole-1-carboxamide was dried in vacuo to give a white solid (0.13 g, 50% yield). ¹H NMR (200 MHz, d₆-DMSO) δ7.05-7.25 (m, 3H), 7.36 (t, J=7.7 Hz, 1H), 7.56 (d, J=7.3 Hz, 1H), 7.79 (d, J=8.7 Hz, 1H), 7.81 (s, 1H), 8.04 (d, J=7.7 Hz, 1H), 8.10 (br s, 1H), 10.92 (s, 1H), 11.88 (s, 1H); ¹³C NMR (50 MHz, d₆-DMSO) δ109.8, 114.5, 120.4, 122.1, 124.1 (2 CH), 124.9, 127.1, 128.0, 129.5, 131.7, 137.6, 148.9, 153.7, 167.1; MS (ES−) m/z 296 (M−H).

### N-(3-Carboxyphenyl)-2,3-dihydro-2-oxo-1H-benzimidazole-1-carboxamide Sodium Salt

N-(3-Carboxyphenyl)-2,3-dihydro-2-oxo-1H-benzimidazole-1-carboxamide (24 mg, 81 mmol) was dispersed in water (2 ml) and sodium bicarbonate (7 mg, 83 mmol) added. Methanol was added dropwise until the compound dissolved and the mixture stirred for 3 h. Concentration under reduced pressure gave N-(3-carboxyphenyl)-2,3-dihydro-2-oxo-1H-benzimidazole-1-carboxamide sodium salt as a white solid.

### EXAMPLE 4

### N-(4-Carboxyphenyl)-2,3-dihydro-2-oxo-1H-benzimidazole-1-carboxamide

2-Hydroxybenzimidazolyl chloroformate (0.20 g, 1.0 mmol) was dissolved in THF (5 ml) and 4-aminobenzoic acid (0.14 g, 1.0 mmol) added. Mixture was shaken at room temperature for 15 h. Solvent was removed in vacuo and the residue triturated with water (3 ml) and ethanol (2×3 ml). The product was dried in vacuo to give N-(4-carboxyphenyl)-2,3-dihydro-2-oxo-1H-benzimidazole-1-carboxamide as an off-white solid (0.12 g, 42% yield). ¹H NMR (200 MHz, d₆-DMSO) δ7.10-7.25 (m, 3H), 7.72 (d, J=8.7 Hz, 2H), 7.96 (d, J=8.7 Hz, 2H), 8.01 (m, 1H), 11.18 (s, 1H), 11.90 (s, 1H); ¹³C NMR (50 MHz, d₆-DMSO) δ109.8, 114.5, 119.1, 120.5, 122.2, 124.2, 126.0, 128.0, 130.8, 141.3, 148.7, 153.7, 166.9; MS (ES−) m/z 296 (M−H).

### EXAMPLE 5

### N-(2-Carboxyphenyl)-2,3-dihydro-2-oxo-1H-benzimidazole-1-carboxamide

2-Hydroxybenzimidazolyl chloroformate (0.20 g, 1.0 mmol) was dissolved in THF (5 ml) and anthranilic acid (0.14 g, 1.0 mmol) added. Mixture was shaken at room temperature for 15 h. Solvent was removed in vacuo and the residue triturated with water (3 ml) and ethanol (2×3 ml). The N-(2-carboxyphenyl)-2,3-dihydro-2-oxo-1H-benzimidazole-1-carboxamide was dried in vacuo to give an off-white solid (0.14 g, 47% yield). ¹H NMR (200 MHz, d₆-DMSO) δ7.04-7.22 (m, 4H), 7.52-7.70 (m, 1H), 7.87-8.05 (m, 2H), 8.39 (d, J=8.4 Hz, 1H), 11.7 (s, 1H), 12.3 (s, 1H); ¹³C NMR (50 MHz, d₆-DMSO) δ109.4, 114.5, 120.5, 121.8, 122.1, 123.5, 124.0, 127.3, 128.4, 131.0, 133.3, 137.0, 149.2, 152.8, 167.9; MS (ES−) m/z 296 (M−H).

### EXAMPLE 6

### N-(3-Carboxymethylphenyl)-2,3-dihydro-2-oxo-1H-benzimidazole-1-carboxamide

2-Hydroxybenzimidazolyl chloroformate (0.20 g, 1.0 mmol) was dissolved in THF (5 ml) and 3-aminophenylacetic acid (0.16 g, 1.0 mmol) added. Mixture was shaken at room temperature for 15 h. Solvent was removed in vacuo and the residue triturated with water (3 ml). Crude product was purified by preabsorption chromatography on silica gel using dichloromethane with ethanol (1 to 10%) followed by ethanol/acetic acid (99:1) as eluent to give N-(3-carboxymethylphenyl)-2,3-dihydro-2-oxo-1H-benzimidazole-1-carboxamide as an off-white solid (0.04 g, 11% yield). ¹H NMR (200 MHz, d₆-DMSO) δ7.03 (d, J=7.6 Hz, 1H), 7.04-7.21 (m, 3H), 7.27-7.38 (m, 1H), 7.43-7.47 (m, 2H), 10.9 (s, 1H), 11.9 (s, 1H); ¹³C NMR (50 MHz, d₆-DMSO) δ40.7, 109.7, 114.5, 118.1, 120.7, 122.1, 124.0, 125.2, 127.1, 128.0, 129.1, 136.1, 137.1, 148.8, 153.8, 172.6; MS (ES−) m/z 310 (M−H).

### EXAMPLE 7

### N-(4-Carboxymethylphenyl)-2,3-dihydro-2-oxo-1H-benzimidazole-1-carboxamide

2-Hydroxybenzimidazolyl chloroformate (0.19 g, 1.0 mmol) was dissolved in THF (5 ml) and 4-aminophenylacetic acid (0.15 g, 1.0 mmol) added. Mixture was shaken at room temperature for 15 h. Solvent was removed in vacuo and the residue triturated with water (3 ml) and ethanol (2×3 ml). N-(4-Carboxymethylphenyl)-2,3-dihydro-2-oxo-1H-benzimidazole-1-carboxamide was dried in vacuo to give an off-white solid (0.10 g, 35% yield). ¹H NMR (200 MHz, d₆-DMSO) δ7.10-7.25 (m, 3H), 7.72 (d, J=8.7 Hz, 2H), 7.96 (d, J=8.7 Hz, 2H), 8.01 (m, 1H), 11.18 (s, 1H), 11.90 (s, 1H); ¹³C NMR (50 MHz, d₆-DMSO) δ40.19, 109.85, 114.56, 119.90, 120.62, 122.17, 124.09, 127.22, 128.04, 130.22, 130.95, 135.80, 148.92, 153.83, 172.94; MS (ES−) m/z 310 (M−H).

### EXAMPLE 8

### N-(4-Ethoxycarbonylphenyl)-2,3-dihydro-2-oxo-1H-benzimidazole-1-carboxamide

2-Hydroxybenzimidazolyl chloroformate (0.44 g, 2.3 mmol) in THF (5 ml) was added to a solution of 4-aminophenylcarboxylate (0.42 g, 2.5 mmol) and triethylamine (0.32 ml, 2.3 mmol) in THF (5 ml). Mixture stirred at room temperature for 15 h. Precipitate was collected by filtration. Crude product was dissolved in THF/water (4:1) and preabsorption chromatography on silica gel was performed using dichloromethane with methanol (0 to 15%) as eluent to give N-(4-ethoxycarbonylphenyl)-2,3-dihydro-2-oxo-1H-benzimidazole-1-carboxamide as a white solid (0.13 g, 18%). Later fractions contained desired product and 2-hydroxybenzimidazole, which was removed by trituration with ethanol. ¹H NMR (200 MHz, d₆-DMSO) δ1.31 (t, J=7.0 Hz, 3H), 4.29 (q, J=7.1 Hz, 2H), 7.10-7.25 (m, 3H), 7.74 (d, J=8.8 Hz, 2H), 7.95-8.10 (m, 3H); ¹³C NMR (50 MHz, d₆-DMSO) δ14.1, 60.5, 109.7, 114.4, 119.0, 122.0, 124.0, 124.9, 126.9, 127.9, 130.4, 141.5, 148.5, 153.6, 165.1; MS (ES−) m/z 324 (M−H).

### EXAMPLE 9

### N-(3-Ethoxycarbonylphenyl)-2,3-dihydro-2-oxo-1H-benzimidazole-1-carboxamide

2-Hydroxybenzimidazolyl chloroformate (0.56 g, 2.8 mmol) in THF (5 ml) was added to a solution of ethyl 3-aminobenzoate (0.52 g, 3.1 mmol) and triethylamine (0.40 ml, 2.9 mmol) in THF (5 ml). Mixture stirred at room temperature for 15 h. Precipitate was removed by filtration. Filtrate was concentrated in vacuo and triturated with ethanol. The crude product was dissolved in dichloromethane/ethanol and preabsorption chromatography on silica gel was performed using dichloromethane as eluent to give N-(4-ethoxycarbonylphenyl)-2,3-dihydro-2-oxo-1H-benzimidazole-1-carboxamide as a white solid (0.11 g, 12%). ¹H NMR (200 MHz, CDCl₃) δ1.44 (t, J=7.0 Hz, 3H), 4.42 (q, J=7.0 Hz, 2H), 7.08-7.28 (m, 3H), 7.45 (t, J=8.0 Hz, 1H), 7.83 (dd, J=1.0, 15.4 Hz, 1H), 8.04 (dd, J=2.1, 8.1 Hz, 1H), 8.18-8.27 (m, 2H); ¹³C NMR (50 MHz, CDCl₃) δ14.3, 61.4, 109.6, 115.9, 121.4, 123.1, 124.3, 125.0, 125.4, 126.9, 127.4, 129.2, 131.1, 137.5, 149.0, 154.2, 166.7; MS (APCI−) m/z 324 (M−H); MS (APCI+) m/z 326 (M+H).

### EXAMPLE 10

### 2-(2,3-Dihydro-2-oxo-benzimidazole-1-carbonyl)-pyrrolidine-1-carboxylic acid tert-butyl ester

Sodium hydride (0.03 g, 1.0 mmol) was added to a solution of 2-hydroxy-benzimidazole (0.11 g, 0.8 mmol) in DMF (5 ml). The mixture was stirred under a nitrogen atmosphere for 90 min. N-t-BOC-L-proline N-hydroxysuccinimide (0.28 g, 0.9 mmol) in DMF (3 ml) was added dropwise and the mixture stirred for 15 h. Solvent was removed in vacuo and the residue partitioned between saturated ammonium chloride solution and ethyl acetate. The organics were separated, dried (MgSO₄), and concentrated under reduced pressure. The crude product was chromatographed on silica gel, petroleum spirit (40-60° C.)/ethyl acetate (7:3) as eluent to give 2-(2,3-dihydro-2-oxo-benzimidazole-1-carbonyl)-pyrrolidine-1-carboxylic acid tert-butyl ester (0.21 g, 78%). The bisacylated compound (0.02 g) was also isolated (determined by MS). The imidazole ring of the product undergoes keto-enol tautomerism, with both forms observed by NMR spectroscopy. When the peaks for each form can be distinguished the frequencies of both forms are listed. ¹H NMR (200 MHz, CDCl₃) δ1.47 and 1.52 (s, 9H), 1.80-2.10 and 2.25-2.55 (m, 4H), 3.38-3.75 (m, 2H), 5.53-5.64 and 5.65-5.67 (dd, J=2 and 7 Hz, 1H), 6.60-6.90 (m, 2H), 7.05-7.18 (m, 1H), 7.62 and 8.15 (d, J=7 Hz, 1H), 9.73 and 10.28 (br s, 1H); ¹³C NMR (50 MHz, CDCl₃) δ23.3 and 23.5, 28.1 and 28.4, 30.0 and 30.7, 46.8 and 47.2, 60.0 and 60.3, 79.8 and 80.3, 109.0 and 109.3, 115.2 and 115.6, 121.8 and 122.5, 124.2 and 124.7, 126.5, 128.5, 152.3 and 152.9, 154.8, 172.5 and 173.8; MS (APCI−) m/z 330 (M−H).

### EXAMPLE 11

### 2-Thioxo-2,3-dihydrobenzoimidazole-1-carboxylic acid phenylamide

Phenyl isocyanate (1 mL, 9.2 mmol) was added to a solution of 2-mercaptobenzimidazole (1.24 g, 8.3 mmol) in DMF (10 mL) and the mixture stirred at room temperature for 15 h. The solvent was removed in vacuo and the residue dispersed in hot methanol (50 mL). The solvent was collected by decanting. On cooling crystals formed and were collected by filtration to give the desired product as a white solid (0.77 g, 35%). ¹H NMR (200 MHz, DMSO-d₆) δ7.17 (t, J=7.2 Hz, 2H), 7.23-7.34 (m, 3H), 7.40 (t, J =7.9 Hz, 3H), 7.61 (d, J=7.8 Hz, 2H), 7.97-8.04 (m, 1H), 12.31 (br s, 1H); ¹³C NMR (50 MHz, DMSO-d₆) δ110.2, 115.0, 119.9, 123.6, 124.7, 125.0, 129.3, 130.6, 131.1, 137.0, 148.3, 167.1; MS (ES−) m/z 268 (M−H).

### EXAMPLE 12

### 1-(Pyrrolidine-2-carbonyl)-1,3-dihydrobenzoimidazol-2-one trifluoroacetic acid salt

Sodium hydride (80% in mineral oil, 48 mg, 1.6 mmol) was added to 2-hydroxybenzimidazole (0.20 g, 1.5 mmol) in DMF (5 mL) and stirred at room temperature, under nitrogen for 90 min. N-t-BOC-L-Proline N-hydroxysuccinimide (0.49 g, 1.6 mmol) dissolved in DMF (5 mL) was added dropwise and the mixture stirred for 15 h. Solvent was removed in vacuo and the residue partitioned between ammonium chloride solution and ethyl acetate. The organics were separated, dried (MgSO₄), and concentrated under reduced pressure to give crude product. Chromatography on silica gel using petroleum spirit (40-60° C.)/ethyl acetate (7:3) as eluent gave N-BOC protected 1-(pyrrolidine-2-carbonyl)-1,3-dihydrobenzoimidazol-2-one as a clear colorless oil (0.33 g, 66%).

N-BOC protected 1-(pyrrolidine-2-carbonyl)-1,3-dihydrobenzoimidazol-2-one was dissolved in dichloromethane (3 mL) and trifluoroacetic acid (6 mL) and stirred at room temperature for 15 h. Solvent was removed in vacuo to give 1-(pyrrolidine-2-carbonyl)-1,3-dihydrobenzoimidazol-2-one trifluoroacetic acid as a glassy solid (0.34 g, 99%). ¹H NMR (200 MHz, DMSO-d₆) δ1.80-2.01 (m, 2H), 2.03-2.23 (m, 1H), 2.26-2.54 (m, 1H), 3.18-3.42 (m, 2H), 5.23-5.42 (m, 1H), 7.04-7.31 (m, 3H), 8.00 (d, J=7.1 Hz, 1H), 8.93 (br s, 1H), 9.47 (br s, 1H); ¹³C NMR (50 MHz, CDCl₃) δ23.9, 29.6, 47.2, 61.2, 110.0, 115.3, 122.8, 125.8, 126.1, 128.6, 152.3, 168.7; MS (APCI+) m/z 232 (M+H).

### EXAMPLE 13

### 2-Oxo-2,3-dihydrobenzoimidazole-1-carboxylic acid allylamide

Allyl isocyanate (0.17 mL, 1.9 mmol) was added to a stirred solution of 2-hydroxybenzimidazole (0.23 g, 1.7 mmol) in DMF (3 mL) and stirred at room temperature for 15 h. The solvent was removed in vacuo and the residue redissolved in methanol (5 mL). Water (1.2 mL) was added dropwise until a precipitate formed. Precipitate was collected by filtration. MS(ES) analysis indicated this was 2-oxo-benzoimidazole-1,3-dicarboxylic acid bis-allylamide (0.11 g, 21%). Repeating the precipitation procedure gave the desired product as a mixture with O-acylation side product.

Triethylamine (20 [symbol] L) was added to 2-oxo-benzoimidazole-1,3-dicarboxylic acid bis-allylamide (41 mg, 0.1 mmol) dissolved in DMSO (1 mL). Mixture was stirred at room temperature for 2 days. Solvent was removed in vacuo and the crude triturated with methanol to give 2-oxo-2,3-dihydro-benzoimidazole-1-carboxylic acid allylamide as a white solid (7 mg, 24%). ¹H NMR (200 MHz, DMSO-d₆) δ3.95 (t, J=5.5 Hz, 2H), 5.06-5.28 (m, 2H), 5.81-6.03 (m, 1H), 7.02-7.20 (m, 3H), 7.96 (d, J=8.2 Hz, 1H), 8.83 (t, J=5.8 Hz, 1H), 11.60 (br s, 1H); ¹³C NMR (50 MHz, DMSO-d₆) δ41.3, 109.4, 114.2, 115.4, 121.7, 123.5, 127.2, 127.9, 134.7, 151.1, 153.6; MS (ES−) m/z 216 (M−H).

### EXAMPLE 14

### 8-Oxo-7,8-dihydropurine-9-carboxylic acid phenylamide

Phenyl isocyanate (35 μL, 0.3 mmol) was added to 7,9-dihydropurin-8-one (40 mg, 0.3 mmol) dissolved in DMF (1 mL). Mixture was stirred at room temperature for 15 h. The solvent was removed in vacuo, the residue triturated with methanol/ethyl acetate/acetic acid (5:2:1) and the precipitate collected by filtration to give 8-oxo-7,8-dihydropurine-9-carboxylic acid phenylamide as a white solid (16 mg, 21%). ¹H NMR (200 MHz, DMSO-d₆) δ7.16 (t, J=6.5 Hz, 2H), 7.40 (t, J=6.9 Hz, 2H), 7.60 (d, J=7.0 Hz, 1H), 8.72 (br s, 1H), 8.92 (br s, 1H), 10.44 (br s, 1H); MS (ES+) m/z 256 (M+H); MS (ES−) m/z 254 (M−H).

### EXAMPLE 15

### 2-Oxo-2,3-dihydrobenzoimidazole-1-carboxylic acid t-butylamide

t-Butyl amine (0.12 mL, 1.1 mmol) was added to 2-hydroxybenzimidazolyl chloroformate (0.21 g, 1.1 mmol) dissolved in THF (5 mL). Mixture was stirred at room temperature for 15 h. Solvent was removed under reduced pressure and the crude product washed with water. Trituration with cold THF, removal of precipitate by filtration, and concentration in vacuo gave 2-oxo-2,3-dihydrobenzoimidazole-1-carboxylic acid t-butylamide as an off-white solid. ¹H NMR (200 MHz, DMSO-d₆) δ1.37 (s, 9H), 7.01-7.15 (m, 3H), 7.98 (d, J=7.7 Hz, 1H), 8.76 (br s, 1H), 10.56 (br s, 1H), 11.67 (br s, 1H); MS (APCI−) m/z 232 (M−H).

### EXAMPLE 16

### 2-Oxo-2,3-dihydrobenzoimidazole-1-carboxylic acid (4-t-butylphenyl)amide

t-Butyl aniline (0.18 mL, 1.1 mmol) was added to 2-hydroxybenzimidazolyl chloroformate (0.20 g, 1.0 mmol) dissolved in THF (5 mL). Mixture was stirred at room temperature for 15 h. Solvent was removed under reduced pressure and the crude product washed with water. The residue was partitioned between ethyl acetate and water. The organic layer was separated, concentrated under reduced pressure to give 2-oxo-2,3-dihydrobenzoimidazole-1-carboxylic acid (4-t-butylphenyl)amide as a tan solid (0.23 g, 71%). ¹H NMR (200 MHz, DMSO-d₆) δ1.26 (s, 9H), 7.05-7.20 (m, 3H), 7.38 (d, J=8.8 Hz, 2H), 7.50 (d, J=8.6 Hz, 2H), 8.03 (d, J=8.1 Hz, 1H), 10.85 (br s, 1H), 11.86 (br s, 1H); ¹³C NMR (50 MHz, DMSO-d₆) δ31.1, 34.1, 109.7, 114.4, 119.6, 122.0, 123.9, 125.8, 127.1, 127.9, 134.6, 146.5, 148.8, 153.7; MS (ES−) m/z 308 (M−H).

### EXAMPLE 17

### 2-Oxo-2,3-dihydrobenzimidazole-1-carboxylic acid phenylamide

Phenyl isocyanate (0.95 mL, 8.7 mmol) was added to a suspension of 2-hydroxybenzimidazole (1.05 g, 7.9 mmol) in DMF (10 mL). Mixture was stirred at room temperature for 15 h. The precipitate was collected by filtration and washed with methanol. The crude product was dissolved in hot DMSO and precipitated by addition of acetic acid to give 2-oxo-2,3-dihydrobenzimidazole-1-carboxylic acid phenylamide as a white solid. ¹H NMR (200 MHz, DMSO-d₆) δ7.08-7.22 (m, 4H), 7.39 (t, J=7.7 Hz, 2H), 7.60 (d, J=7.6 Hz, 2H), 8.03 (d, J=7.2 Hz, 1H), 10.94 (br s, 1H); ¹³C NMR (50 MHz, DMSO-d₆) δ31.1, 34.1, 109.7, 114.4, 119.6, 122.0, 123.9, 125.8, 127.1, 127.9, 134.6, 146.5, 148.8, 153.7; MS (APCI−) m/z 252 (M−H).

### EXAMPLE 18

### 2-Oxo-2,3-dihydroimidazole[4,5-c]pyridine-1-carboxylic acid m-tolylamide

m-Tolyl isocyanate (0.12 mL, 0.9 mmol) was added dropwise to a stirred suspension of 1,3-dihydroimidazo[4,5-c]pyridin-2-one (0.10 g, 0.7 mmol) in DMF (2 mL). Mixture was stirred at room temperature for 17 h. The precipitate was collected by filtration and washed with DMF to give 2-oxo-2,3-dihydroimidazole[4,5-c]pyridine-1-carboxylic acid m-tolylamide as a white solid (0.11 g, 57%). ¹H NMR (200 MHz, DMSO-d₆) δ2.32 (s, 3H), 6.98 (d, J=7.7 Hz, 1H), 7.20 (d, J=5.5 Hz, 1H), 7.22-7.32 (m, 1H), 7.38-7.45 (m, 2H), 8.31 (d, J=5.5 Hz, 1H), 9.00 (s, 1H), 10.80 (s, 1H), 12.50 (br s, 1H); MS (APCI+) m/z 269 (M+H); MS (APCI−) m/z 267 (M−H).

### EXAMPLE 19

### 2,2-Dioxo-2,3-dihydro-2λ⁶-benzo[1,2,5]thiadiazole-1-carboxylic acid (3,5-dimethoxyphenyl)amide

3,5-Dimethoxyphenyl isocyanate (51 mg, 0.28 mmol), 1,3-dihydrobenzo[1,2,5]thiadiazole-2,2-dioxide (38 mg, 0.22 mmol), and triethyl amine (50 μL, 0.36 mmol) were dissolved in THF and heated at reflux for 16 h. Solvent was removed in vacuo and the crude product purified by chromatography on silica gel (5% methanol in dichloromethane as eluent) to give 2,2-dioxo-2,3-dihydro-2λ⁶-benzo[1,2,5]thiadiazole-1-carboxylic acid (3,5-dimethoxyphenyl)amide as a fawn colored oil. ¹H NMR (200 MHz, CDCl₃) δ3.79 (s, 6H), 6.25 (t, J=2.4 Hz, 1H), 6.75 (d, J=2.2 Hz, 2H), 6.83-7.00 (m, 4H), 7.83-7.88 (m, 1H), 8.23 (s, 1H); MS (APC+) m/z 350 (M+H); MS (APCI−) m/z 348 (M−H).

### EXAMPLE 20

### 2-Oxo-2,3-dihydrobenzimidazole-1-carboxylic acid (1,1,3-trioxo-2,3-dihydro-1H-1λ⁶-benzo [d]isothiazol-5-yl)amide

2-Hydroxybenzimidazolyl chloroformate (0.20 g, 1.0 mmol) was dispersed in THF (10 mL) and 6-aminosaccharin (0.19 g, 1.0 mmol) added. 6-Aminosaccharin was prepared by a literature procedure (Kamogawa, H. et al., Bull. Chem. Soc. Jpn. 55: 3824-3827, 1982). Mixture was stirred at room temperature for 15 h. The precipitate was collected by filtration, washed with THF, and dried to give 2-oxo-2,3-dihydrobenzimidazole-1-carboxylic acid (1,1,3-trioxo-2,3-dihydro-1H-1λ⁶-benzo[d]isothiazol-5-yl)amide as a white powder (0.07 g, 20%). ¹H NMR (200 MHz, d₆-DMSO) δ7.11-7.20 (m, 3H), 8.00 (s, 2H), 8.04 (d, J=6.1 Hz, 1H), 8.48 (s, 1H); MS (APCI−) m/z 357 (M−H).

### EXAMPLE 21

### 2-Hydroxy-5-[(2-oxo-2,3-dihydrobenzimidazole-1-carbonyl)-amino]benzoic acid

2-Hydroxybenzimidazolyl chloroformate (0.20 g, 1.0 mmol) was dispersed in THF (10 mL) and 5-amino-2-hydroxybenzoic acid (0.16 g, 1.1 mmol) added. Mixture was stirred at room temperature for 15 h. Mixture was concentrated under reduced pressure and the residue washed with ethanol to give 2-hydroxy-5-[(2-oxo-2,3-dihydrobenzimidazole-1-carbonyl)-amino]benzoic acid as a white powder (0.12 g, 37%). ¹H NMR (200 MHz, d₆-DMSO) δ7.01 (d, J=7.7 Hz, 1H), 7.10-7.28 (m, 3H), 7.66 (dd, J=7.7, 2.6 Hz, 1H), 8.04 (d, J=6.5 Hz, 1H), 8.12 (d, J=2.6 Hz, 1H), 10.80 (s, 1H), 8.10 (s, 1H), 11.88 (s, 1H); MS (APCI−) m/z 312 (M−H).

### EXAMPLE 22

### 4-{[(2-Oxo-2,3-dihydrobenzimidazole-1-carbonyl)-amino]-methyl}benzoic acid

2-Hydroxybenzimidazolyl chloroformate (0.20 g, 1.0 mmol) was dispersed in THF (13 mL) and 4-(aminomethyl)benzoic acid (0.16 g, 1.1 mmol) added. Mixture was stirred at room temperature for 15 h. Mixture was concentrated under reduced pressure and the residue washed with water. The crude product was purified by chromatography on silica gel (7% methanol in dichloromethane gradient to 100% methanol as eluent) to give 4-{[(2-oxo-2,3-dihydrobenzimidazole-1-carbonyl)-amino]-methyl}benzoic acid as a grey powder (0.03 g, 11%). ¹H NMR (200 MHz, d₆-DMSO) δ4.54 (d, J=5.8 Hz, 2H), 7.00-7.19 (m, 3H), 7.28 (d, J=7.1 Hz, 1H), 7.83 (d, J=7.1 Hz, 2H), 7.99 (d, J=7.7 Hz, 1H), 9.30 (br s, 1H); MS (APCI−) m/z 310 (M−H).

### EXAMPLE 23

### 5- [(2-Oxo-2,3-dihydrobenzimidazole-1-carbonyl)-amino]isophthalic acid

2-Hydroxybenzimidazolyl chloroformate (0.20 g, 1.0 mmol) was dispersed in THF (17 mL) and 5-aminoisophthalic acid (0.21 g, 1.2 mmol) added. Mixture was stirred at room temperature for 15 h. The precipitate was collected by filtration, washed with ethanol and water to give 5-[(2-oxo-2,3-dihydrobenzimidazole-1-carbonyl)-amino]isophthalic acid as a cream solid (0.19 g, 52%). ¹H NMR (200 MHz, d₆-DMSO) δ7.10-7.31 (m, 3H), 8.06 (d, J=6.9 Hz, 1H), 8.25 (s, 1H), 8.40 (s, 2H), 11.20(s, 1H), 11.95 (br s, 1H); MS (APCI−) m/z 340 (M−H).

### EXAMPLE 24

### 2-Oxo-2,3-dihydrobenzimidazole-1-carboxylic acid (4-sulfamoylphenyl)amide

2-Hydroxybenzimidazolyl chloroformate (0.12 g, 0.6 mmol) was dispersed in THF (11 mL) and sulfanilamide (0.11 g, 0.6 mmol) added. Mixture was stirred at reflux for 15 h. Mixture was concentrated under reduced pressure and the residue washed with ethanol to give 2-oxo-2,3-dihydrobenzimidazole-1-carboxylic acid (4-sulfamoylphenyl)amide as a pale yellow solid (0.11 g, 54%). ¹H NMR (200 MHz, d₆-DMSO) δ7.10-7.29 (m, 3H), 7.37 (s, 2H), 7.76-7.93 (m, 4H), 8.08 (d, J=7.7 Hz, 1H), 11.21(s, 1H); MS (APCI−) m/z 331 (M−H).

### EXAMPLE 25

### 2-Oxo-2,3-dihydrobenzimidazole-1-carboxylic acid (3-chlorophenyl)amide

2-Hydroxybenzimidazolyl chloroformate (0.19 g, 1.0 mmol) was dispersed in THF (10 mL) and 3-chloroaniline (0.13 g, 1.0 mmol) added. Mixture was stirred at room temperature for 15 h. The precipitate was collected by filtration, washed with THF. The crude product was purified by chromatography on silica gel (5% methanol in dichloromethane as eluent) to give 2-oxo-2,3-dihydrobenzimidazole-1-carboxylic acid (3-chlorophenyl)amide as a pale yellow solid (0.10 g, 38%). ¹H NMR (200 MHz, d₆-DMSO) δ7.08-7.31 (m, 4H), 7.39-7.55 (m, 2H), 7.87 (s, 1H), 8.04 (d, J=7.1 Hz, 1H), 11.08 (s, 1H); MS (APCI−) m/z 286 (M−H).

### EXAMPLE 26

### Phosphoric acid mono-{4[(2-oxo-2,3-dihydrobenzimidazole-1-carbonyl)-amino]-phenyl} ester

4-Nitrophenylphosphate disodium salt hexahydrate (2.01 g, 5.4 mmol) was dissolved in water (50 mL) and 10% palladium on carbon (0.21 g) was added. The mixture was stirred under an atmosphere of hydrogen for 15 h. The carbon was removed by filtration and the filtrate freeze dried to give 4-aminophenylphosphate disodium salt as a white solid (1.2 g, 95%).

2-Hydroxybenzimidazolyl chloroformate (0.20 g, 1.0 mmol) was dispersed in THF (15 mL) and 4-aminophenylphosphate disodium salt (0.26 g, 1.1 mmol) added. Mixture was stirred at room temperature for 15 h. The precipitate was collected by filtration and washed with THF. The crude product was dissolved in water, ion-exchanged (IR120 H+ form), and freeze dried to give phosphoric acid mono-{4[(2-oxo-2,3-dihydrobenzimidazole-1-carbonyl)-amino]-phenyl} ester as a cream solid (0.07 g, 19%). ¹H NMR (200 MHz, d₆-DMSO) δ7.03-7.31 (m, 5H), 7.52 (d, J=7.5 Hz, 1H), 7.60 (d, J=7.5 Hz, 1H), 8.04 (d, J=7.5 Hz, 1H), 10.88 (d, J=5.3 Hz, 1H), 11.86 (d, J=15.1 Hz, 1H); MS (APCI−) m/z 348 (M−H).

### EXAMPLE 27

### 2-Oxo-2,3-dihydrobenzimidazole-1-carboxylic acid biphenyl-4-ylamide

2-Hydroxybenzimidazolyl chloroformate (0.20 g, 1.0 mmol) was dispersed in THF (13 mL) and 4-aminobiphenyl (0.19 g, 1.1 mmol) added. Mixture was stirred at room temperature for 15 h. Mixture was concentrated under reduced pressure. The crude product was purified by chromatography on silica gel (5% methanol in dichloromethane as eluent) to give 2-oxo-2,3-dihydrobenzimidazole-1-carboxylic acid biphenyl-4-ylamide as a white solid (0.08 g, 25%). ¹H NMR (200 MHz, d₆-DMSO) δ7.10-7.24 (m, 3H), 7.35-7.52 (m, 3H), 7.73 (m, 6H), 8.07 (dd, J=7.2, 2.1 Hz, 1H), 11.06 (br s, 1H); MS (APCI−) m/z 328 (M−H).

### EXAMPLE 28

### N-(Phenyl-4-boronic acid) -2,3-dihydro-2-oxo-1H-benzimidazole-1-carboxamide

2-Hydroxybenzimidazolyl chloroformate (0.30 g, 1.5 mmol) was dispersed in THF (15 mL) and 4-(4,4,5,5-tetramethyl-1,3,2-dioxaborolan-2-yl)aniline (0.38 g, 1.7 mmol) added. Mixture was stirred at room temperature for 15 h. Mixture was concentrated under reduced pressure and triturated with ethanol to give N-(phenyl-4-boronic acid) pinacol ester 2,3-dihydro-2-oxo-1H-benzimidazole-1-carboxamide as a creamy white solid (0.17 g, 30%).

The N-(phenyl-4-boronic acid) pinacol ester (0.17 g, 0.5 mmol) was suspended in 6N HCl (10 mL) and heated at reflux for 2 h. On cooling the precipitate was collected by filtration to give N-(phenyl-4-boronic acid)-2,3-dihydro-2-oxo-1H-benzimidazole-1-carboxamide as a off-white solid (0.11 g, 85%). ¹H NMR (200 MHz, d₆-DMSO) δ7.08-7.29 (m, 3H), 7.60 (d, J=7.2 Hz, 2H), 7.84 (d, J=7.2, Hz, 2H), 7.98-8.13 (m, 3H), 11.05 (br s, 1H); MS (ES−) m/z 296 (M−H).

### EXAMPLE 29

### 2-Oxo-2,3-dihydrobenzimidazole-1-carbothioic acid (3-trifluoromethylphenyl)amide

3-(Trifluoromethyl)phenylisothiocyanate (1.4 mL, 9.3 mmol) was added dropwise to a stirred solution of 1,2-phenylenediamine (1.0 g, 9.3 mmol) in benzene. The mixture was stirred under nitrogen at room temperature for 15 h. Precipitate was collected by filtration and washed with toluene to give 1-(2-aminophenyl)-3-(3-trifluoromethylphenyl)thiourea as a pale pink solid.

A mixture of N,N′-disuccinimidyl carbonate (0.17 g, 0.7 mmol) and acetonitrile (6 mL) was added dropwise to a solution of 1-(2-aminophenyl)-3-(3-trifluoromethylphenyl)thiourea (0.20 g, 0.6 mmol) in acetonitrile (10 mL). The mixture was stirred under nitrogen at room temperature for 18 h. The resulting suspension was filtered and washed with acetonitrile to give 2-oxo-2,3-dihydrobenzimidazole-1-carbothioic acid (3-trifluoromethylphenyl)amide as a white solid (0.10 g, 48%). ¹H NMR (200 MHz, d₆-DMSO) δ7.00-7.25 (m, 3H), 7.60-7.78 (m, 2H), 7.94-8.08 (br m, 1H), 8.10-8.20 (br m, 1H), 8.25 (br s, 1H), 12.10 (br s, 1H); MS (APCI−) m/z 336 (M−H).

### EXAMPLE 30

### 2-Oxo-2.3-dihydrobenzimidazole-1-carbothioic acid p-tolylamide

p-Tolylisothiocyanate (1.28 g, 8.6 mmol) was added to a stirred solution of 1,2-phenylenediamine (1.0 g, 9.3 mmol) in benzene (25 mL). The mixture was stirred under nitrogen at room temperature for 15 h. Precipitate was collected by filtration and washed with toluene to give 1-(2-aminophenyl)-3-p-tolylthiourea as a pale pink solid.

N,N′-Disuccinimidyl carbonate (0.20 g, 0.8 mmol) was added to a solution of 1-(2-aminophenyl)-3-p-tolylthiourea (0.20 g, 0.8 mmol) in acetonitrile (8 mL). The mixture was stirred under nitrogen at room temperature for 16 h. The resulting suspension was filtered and washed with acetonitrile to give 2-oxo-2,3-dihydrobenzimidazole-1-carbothioic acid p-tolylamide as a white solid (0.08 g, 37%). ¹H NMR (200 MHz, d₆-DMSO) δ2.33 (s, 3H), 7.03-7.22 (m, 3H), 7.26 (d, J 8.4 Hz, 2H), 7.65 (d, J=8.4 Hz, 2H), 8.10-8.16 (m, 1H), 11.90 (br s, 1H); MS (APCI−) m/z 282 (M−H).

### EXAMPLE 31

### 2-Thioxo-2,3-dihydrobenzimidazole-1-carbothioic acid (3-trifluoromethlphenyl) amide

1-(2-Aminophenyl)-3-(3-trifluoromethylphenyl)thiourea (0.20 g, 0.6 mmol) was added to a stirred solution of 1,1′-thiocarbonyldiimidazole (0.13 g, 0.7 mmol) in benzene (5 mL). The mixture was stirred under nitrogen at room temperature for 15 h. The resulting suspension was filtered and washed with benzene to give 2-thioxo-2,3-dihydrobenzimidazole-1-carbothioic acid (3-trifluoromethylphenyl)amide as a cream solid. ¹H NMR (200 MHz, d₆-DMSO) δ7.10-7.28 (m, 3H), 7.42-7.48 (m, 1H), 7.70-7.80 (m, 2H), 8.14-8.20 (m, 1H), 8.50 (br s, 1H), 9.12 (t, J=1.1 Hz, 2H); MS (APCI−) m/z 352 (M−H).

### EXAMPLE 32

### 2-Thioxo-2,3-dihydrobenzimidazole-1-carbothioic acid p-tolylamide

1-(2-Aminophenyl)-3-p-tolylthiourea (0.20 g, 0.8 mmol) was added to a stirred solution of 1,1′-thiocarbonyldiimidazole (0.15 g, 0.9 mmol) in benzene (5 mL). The mixture was stirred under nitrogen at room temperature for 15 h. The resulting suspension was filtered and washed with benzene to give 2-thioxo-2,3-dihydrobenzimidazole-1-carbothioic acid p-tolylamide as a pale yellow solid. ¹H NMR (200 MHz, d₆-DMSO) δ2.34 (s, 3H), 7.18-7.33 (m, 3H), 7.29 (d, J=8.4 Hz, 2H), 7.35-7.44 (m, 1H), 7.85 (d, J=8.4 Hz, 2H), 9.10 (s, 1H); MS (APCI−) m/z 298 (M−H).

### EXAMPLE 33

### 2-Oxo-2,3-dihydrobenzimidazole-1-carboxylic acid cyclohexylamide

Cyclohexyl isocyanate (2.4 mL, 18.8 mmol) was added to a stirred solution of 1,2-phenylenediamine (2.0 g, 18.5 mmol) in benzene (20 mL). The mixture was stirred under nitrogen at reflux for 3 h. Precipitate was collected by filtration and washed with benzene to give 1-(2-aminophenyl)-3-cyclohexyl urea as a beige solid.

N,N′-Disuccinimidyl carbonate (0.25 g, 1.0 mmol) was added to a solution of 1-(2-aminophenyl)-3-cyclohexyl urea (0.20 g, 0.9 mmol) in acetonitrile (6 mL). The mixture was stirred under nitrogen at room temperature for 18 h. The resulting suspension was filtered and washed with acetonitrile to give as a white solid (0.15 g, 68%). ¹H NMR (200 MHz, d₆-DMSO) δ1.10-1.95 (m, 10H), 3.60-3.80 (m, 1H), 7.02-7.18 (m, 2H), 7.95-8.01 (m, 1H), 8.74 (d, J=7.3 Hz, 1H), 11.50 (br s, 1H); MS (APCI−) m/z 258 (M−H).

### EXAMPLE 34

### 5-Methoxy-2-oxo-2,3-dihydrobenzimidazole-1-carboxylic acid phenylamide

N,N′-Disuccinimidyl carbonate (0.06 g, 0.2 mmol) was added to a solution of 1-(2-amino-4-methoxyphenyl)-3-phenyl urea (0.06 g, 0.2 mmol) in acetonitrile (2 mL). The mixture was stirred under nitrogen at room temperature for 16 h. The resulting suspension was filtered and washed with acetonitrile to give 5-methoxy-2-oxo-2,3-dihydrobenzimidazole-1-carboxylic acid phenylamide as a white solid (0.03 g, 48%). ¹H NMR (200 MHz, d₆-DMSO) δ3.80 (s, 3H), 6.68-6.80 (m, 2H), 7.10-7.22 (m, 1H), 7.36-7.45 (m, 2H), 7.58-7.65 (m, 2H), 7.95 (d, J=8.0 Hz, 1H), 10.85 (br s, 1H), 11.83 (br s, 1H).

### EXAMPLE 35

### 2-Oxo-2,3-dihydrobenzimidazole-1-carboxylic acid (4,6-dimethylpyrimidin-2-yl) amide

2-Hydroxybenzimidazolyl chloroformate (0.50 g, 2.6 mmol) was dispersed in THF (20 mL) and 2-amino-4,6-dimethylpyrimidine (0.31 g, 2.6 mmol) added. Mixture was stirred at room temperature for 36 h. The precipitate was collected by filtration and washed with THF to give 2-oxo-2,3-dihydrobenzimidazole-1-carboxylic acid (4,6-dimethylpyrimidin-2-yl) amide as a cream solid (0.17 g, 24%). ¹H NMR (200 MHz, d₆-DMSO) δ2.40 (s, 6H), 7.03 (s, 1H), 7.09-7.26 (m, 3H), 8.03-8.09 (m, 1H), 11.45 (s, 1H); MS (APCI+) m/z 284 (M+H).

### EXAMPLE 36

### Rat Aorta Assay for Angiogenesis Inhibition

The rat aorta ring model based on that described by Nicosia and Ottinetti (Nicosia, R. F. et al., Lab. Investigation 63: 115, 1990; Nicosia, R. F. et al., Cell. Dev. Bio. 26: 119-128, 1990), was used throughout the assay.

The agarose was made up as a 1.5% solution in distilled water and brought to the boil to form a clear solution which was poured into sterile 9 cm petri dishes, covered and allowed to cool and set.

Maintaining sterile conditions, agarose rings were obtained by punching two concentric circles, with sterile 10 and 17 mm hole punches, respectively, in the agarose gel. Using sterile forceps, the rings are removed and placed, three per well in each of the 6-well plates.

The MEM was prepared according to manufacturer's directions, but before filtering through a 0.22 μm filter, HEPES and L-glutamine were added to give 10 mM and 1 mM concentrations respectively with pH adjusted to 7.4. Eight hundred mL of this medium were filtered through a 0.2 μm filter along with the antibiotics (50 mg/L Gentamycin sulphate and 2.5 mg/L Amphotericin B) and 200 mL of FCS (to give 20%) to yield one liter of medium.

The aorta was removed from a 3-4 month male Copenhagen rat and transferred to a dissecting dish where it was cleaned and carefully stripped of the fibroadipose tissue surrounding it. Rings of 0.5 mm were cut, using a fresh scalpel blade, from the length of the aorta. These were kept under sterile conditions in a biohazard hood where they were washed 12 times with MEM.

Before transferring the aortic rings to the culture plate, the bottom of each agarose well was coated with 150 μL of clotting fibrinogen. Fibrinogen was made up as a 3 mg/L solution in MEM, while Thrombin made up in distilled water to give a concentration of 50 U/mL. The fibrinogen (1 mL) and the thrombin (20 μL) reacted within 30 sec to form a solid gel.

The aortic rings were transferred to the 6-well plates, with one ring placed in the center of each agarose well. Fresh fibrinogen/thrombin was made up as before and 150 μL was used to seal in each aortic ring. The gels were rested for approximately 2 hours before the medium was added.

The test compounds were prepared to give three concentration for testing −4, 2 and 100 μg/mL. The compounds were made up as 6 mg/mL solution in water or DMSO. The test solutions were added to each well with the medium.

Six mL of MEM were carefully added to each of three wells to become the controls. MEM, along with the test compounds were added to the remaining wells and all were covered and transferred to the CO₂ Incubator at 37° C., where they were kept for the next 14 days.

The plates were checked each day, but very little growth was observed in the first 4 days. However, by the fifth day, there were noticeable changes in the tissue. Micro-vessels were seen and scored. We have based the scoring method on that used by Liekens et al., (Liekins, S. et al., Oncol. Res. 9: 173-181, 1997) in which 0 meant no vessels and 10 meant maximum vessels; the score is then converted into a percentage inhibition of vessel growth.

Results

| | Concentration | Growth Inhibition | Growth Inhibition |
|---|---|---|---|
| Example | (μg/ml) | Day 5 (%) | Day 11 (%) |
| 2 | 100 | * | 100* |
| | 50 | 100 | 100 |
| | 20 | 100 | 100 |
| | 4 | 50 | 0 |
| 3 | 100 | 60 | 60 |
| Sodium salt of 3 | 100 | 70 | 80 |
| 4 | 100 | * | 100* |
| | 50 | 90 | 95 |
| | 20 | 100 | 70 |
| 5 | 100 | * | 90* |
| | 50 | 10 | 0 |
| | 20 | 0 | 0 |
| 7 | 100 | * | 100* |
| | 50 | 80 | 100 |
| | 20 | 80 | 0 |
| 9 | 100 | 70 | 10 |
| 11 | 50 | 100 | |
| | 4 | 0 | |
| 12 | 20 | 70 | |
| | 4 | 50 | |
| 13 | 20 | 20 | |
| | 4 | 0 | |
| 14 | 50 | 20 | |
| | 20 | 40 | |
| | 4 | 20 | |
| 16 | 10 | 10 | |
| 17 | 50 | 95 | |
| | 20 | 30 | |
| | 4 | 50 | |
| 20 | 10 | 100 | 70 |
| 23 | 10 | 10 | |
| 26 | 10 | 90 | 40 |
| 28 | 10 | | 20 |
| | 4 | | 20 |

*Test slow to establish. Growth inhibition after 15 days.

What we claimed is:

1. A compound of the formula: 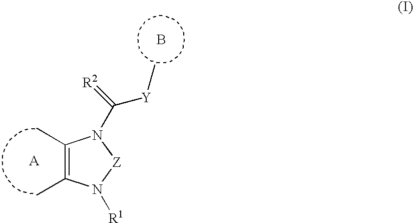
and pharmaceutically acceptable salts thereof, wherein:
Y is a direct bond or a linker group selected from a group of CH₂, NH, NR₁, S, SO, SO₂, or O;
Z is CO, CS, SO, SO₂, or C═NH;
R¹ is H, alkyl, alkenyl, alkynyl, cycloalkyl, aryl, arylalkyl, acyl, alkoxyacyl, aryloxyacyl, or aminoacyl groups;
R² is O, S, or NH;
A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy and n is 0-11; and
B is alkyl, arylalkyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; excluding the following compounds:
(i) a compound, wherein Y is a direct bond, Z is CO, R¹ is 1-(piperidin-4-yl)piperidin-4-yl, R² is O, A is phenyl, and B is methyl;
(ii) compounds, wherein Y is NH, Z is CO, R¹ is H, R² is O, A is phenyl, and B is phenyl, bromophenyl, mono/dichlorophenyl, methoxyphenyl, tolyl, nitrophenyl, 3-trifluoromethylphenyl, benzoic acid, phenylsulfonylaminophenyl, 1-naphthalenyl, 4-piperidinyl, azabicyclooctyl, or azabicyclononyl;
(iii) compounds, wherein Y is NH, Z is CO, R¹ is H, R² is O, A is nitrophenyl, and B is phenyl, 4-bromophenyl, or 3,4-dichlorophenyl;
(iv) compounds, wherein Y is NH, Z is CO, R¹ is H, R² is O, A is mono/dichlorophenyl, and B is 4-bromophenyl, phenyl, dichlorophenyl, mono/di alkoxyphenyl, 1-piperidinyl, or isoindol-5-yl;
(v) compounds, wherein Y is NH, Z is CO, R¹ is H, R² is O, A is mono/dimethylphenyl, and B is phenyl or methylphenyl;
(vi) a compound, wherein Y is NH, Z is CO, R¹ is H, R² is O, A is naphthalenyl, and B is phenyl;
(vii) compounds, wherein Y is NH, Z is CO, R² is O, A is phenyl, R¹ is i-propyl, and B is 1-piperazinyl, 4-pyridinyl, 4-piperidinyl, or methanopyrolizin-1-yl;
(viii) compounds, wherein Y is NH, Z is CO, R² is O, A is phenyl, R¹ is alkyl, alkenyl, or alkynyl, and B is azabicyclooctyl, azabicyclononyl, or azabicyclodecyl;
(ix) a compound, wherein Y is NMe, Z is CO, R² is O, R¹ is H, A is phenyl, and B is azabicyclononyl;
(x) a compound, wherein Y is NPh, Z is CO, R² is O, R¹ is H, and A and B are phenyl;
(xi) a compound, wherein Y is NR, R is dimethoxyphenylmethyl, Z is CO, R² is O, R¹ is H, A is phenyl, and B is azabicyclononyl;
(xii) a compound, wherein Y is NPh, Z is CO, R² is O, B is phenyl, R¹ and A taken together to form guanosine derivatives; and
(xiii) compounds, wherein Y is a NH, Z is CS, R¹ is H, R² is S, A is phenyl, and B is aminophenyl, benzoic acid, benzyl, butyl, hydroxyphenyl, chlorophenyl, methyl, morpholinyl, propyl, phenyl, pyridinyl, or thiophene-2-carboxylate.

2. The compound of claim 1, wherein A and B are cycloalkyl or aryl ring groups, present as a single ring, or condensed with other ring, wherein each ring is selected from the group consisting of cyclopropane, cyclobutane, cyclopentane, cycloheptane, cyclohexane, cyclohexanedione, cyclopentanedione, quinone, tricyclododecane, pyrrolidine, piperidine, piperazine, morpholine, thiophene, pyrrole, pyrazole, imidazole, 1,2,3-triazole, 1,2,4-triazole, oxazole, isoxazole, thiazole, isothiazole, furan, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, benzene, pyridine, pyridazine, pyrimidine, pyrazine, triazine, indene, naphthalene, indole, isoindole, indolizine, benzofuran, benzothiophene, indazole, benzimidazole, benzthiazole, purine, quinolizine, quinoline, isoquinoline, cinnoline, phthalazine, quinazoline, quinoxaline, naphthyridine, pteridine, fluorene, carbazole, carboline, acridine, phenazine, and anthracene, optionally substituted at one or more positions with alkyl, alkoxy, aryl, aryloxy, alkaryl, alkaryloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy; and n is 0-11.

3. The compound of claim 1, wherein Y is a direct bond, Z is CO, R² is O, and A, B, and R¹ are as defined in claim 1.

4. The compound of claim 1, wherein Y is NH, Z is CO, R² is O, and A, B, and R¹ are as defined in claim 1.

5. The compound of claim 1, wherein Y is a NH, Z is CS, R² is O, and A, B, and R¹ are as defined in claim 1.

6. The compound of claim 1, wherein Y is a NH, Z is SO₂, R² is O, and A, B, and R¹ are as defined in claim 1.

7. The compound of claim 1, wherein Y is a NH, Z is CO, and R² is S, and A, B, and R¹ are as defined in claim 1.

8. The compound of claim 1, wherein Y is a NH, Z is CS, and R² is S, and A, B, and R¹ are as defined in claim 1.

9. The compound of claim 1, wherein the compound is selected from the group consisting of N-(4-Nitrophenyl)-2,3-dihydro-2-oxo-1H-benzimidazole-1-carboxamide;
N-(Phenyl-3-boronic acid)-2,3-dihydro-2-oxo-1H-benzimidazole-1-carboxamide;
N-(3-Carboxyphenyl)-2,3-dihydro-2-oxo-1H-benzimidazole-1-carboxamide;
N-(3-Carboxyphenyl)-2,3-dihydro-2-oxo-1H-benzimidazole-1-carboxamide Sodium salt;
N-(4-Carboxyphenyl)-2,3-dihydro-2-oxo-1H-benzimidazole-1-carboxamide;
N-(2-Carboxyphenyl)-2,3-dihydro-2-oxo-1H-benzimidazole-1-carboxamide;
N-(3-Carboxymethylphenyl)-2,3-dihydro-2-oxo-1H-benzimidazole-1-carboxamide;
N-(4-Carboxymethylphenyl)-2,3-dihydro-2-oxo-1H-benzimidazole-1-carboxamide;
N-(4-Ethoxycarbonylphenyl)-2,3-dihydro-2-oxo-1H-benzimidazole-1-carboxamide;
N-(3-Ethoxycarbonylphenyl)-2,3-dihydro-2-oxo-1H-benzimidazole-1-carboxamide;
2-(2,3-Dihydro-2-oxo-benzimidazole-1-carbonyl)-pyrrolidine-1-carboxyamide;
2-thioxo-2,3-dihydrobenzoimidazole-1-carboxylic acid phenylamide;
1-(pyrrolidine-2-carbonyl)-1,3-dihydrobenzoimidazo 1-2-one trifluoroacetic acid salt;
2-oxo-2,3-dihydrobenzoimidazole-1-carboxylic acid allylamide;
8-oxo-7,8-dihydropurine-9-carboxylic acid phenylamide;
2-oxo-2,3-dihydrobenzoimidazole-1-carboxylic acid t-butylamide;
2-oxo-2,3-dihydrobenzoimidazole-1-carboxylic acid (4-t-butylphenyl) amide;
2-oxo-2,3-dihydrobenzimidazole-1-carboxylic acid phenylamide;
2-oxo-2,3-dihydroimidazole[4,5-c]pyridine-1-carboxylic acid m-tolylamide;
2,2-dioxo-2,3-dihydro-2λ⁶-benzo[1,2,5]thiadiazole-1-carboxylic acid
(3,5-dimethoxyphenyl)amide;
2-oxo-2,3-dihydrobenzimidazole-1-carboxylic acid (1,1,3-trioxo-2,3-dihydro-1H-1λ⁶-benzo[d]isothiazol-5-yl)amide; p1 2-hydroxy-5-[(2-oxo-2,3-dihydrobenzimidazole-1-carbonyl)-amino]benzoic acid;
4-{[(2-oxo-2,3-dihydrobenzimidazole-1-carbonyl)-amino]-methyl}benzoic acid;
5-[(2-oxo-2,3-dihydrobenzimidazole-1-carbonyl)-amino]isophthalic acid;
2-oxo-2,3-dihydrobenzimidazole-1-carboxylic acid (4-sulfamoylphenyl)amide;
2-oxo-2,3-dihydrobenzimidazole-1-carboxylic acid (3-chlorophenyl)amide;
phosphoric acid mono-{4[(2-oxo-2,3-dihydrobenzimidazole-1-carbonyl)-amino]-phenyl} ester;
2-oxo-2,3-dihydrobenzimidazole-1-carboxylic acid biphenyl-4-ylamide;
N-(phenyl-4-boronic acid)-2,3-dihydro-2-oxo-1H-benzimidazole-1-carboxamide;
2-oxo-2,3-dihydrobenzimidazole-1-carbothioic acid (3-trifluoromethylphenyl)amide;
2-oxo-2,3-dihydrobenzimidazole-1-carbothioic acid p-tolylamide;
2-thioxo-2,3-dihydrobenzimidazole-1-carbothioic acid (3-trifluoromethylphenyl) amide;
2-thioxo-2,3-dihydrobenzimidazole-1-carbothioic acid p-tolylamide;
2-oxo-2,3-dihydrobenzimidazole-1-carboxylic acid cyclohexylamide;
5-methoxy-2-oxo-2,3-dihydrobenzimidazole-1-carboxylic acid phenylamide; and
2-oxo-2,3-dihydrobenzimidazole-1-carboxylic acid (4,6-dimethylpyrimidin-2-yl) amide.

10. The compound of claim 1, wherein the compound is selected from the group consisting of N-(phenyl-3-boronic acid)-2,3-dihydro-2-oxo-1H-benzimidazole-1-carboxamide; N-(4-carboxyphenyl)-2,3-dihydro-2-oxo-1H-benzimidazole-1-carboxamide;
2-oxo-2,3-dihydrobenzimidazole-1-carboxylic acid (1,1,3-trioxo-2,3-dihydro-1H-1λ⁶⁻benzo[d]isothiazol-5-yl)amide; phosphoric acid mono-{4[(2-oxo-2,3-dihydrobenzimidazole-1-carbonyl)-amino]-phenyl} ester; and N-(phenyl-4-boronic acid)-2,3-dihydro-2-oxo-1H-benzimidazole-1-carboxamide.

11. A pharmaceutical composition comprising a pharmaceutically acceptable carrier or excipient and a therapeutically effective amount of a compound of the formula: 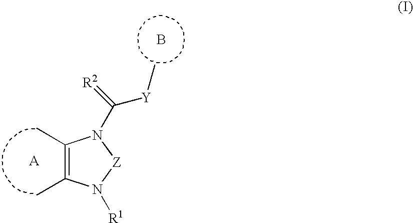
or pharmaceutically acceptable salts thereof, wherein:
Y is a direct bond or a linker group selected from a group of CH₂, NH, NR₁, S, SO, SO₂, or O;
Z is CO, CS, SO, SO₂, or C═NH;
R¹ is H, alkyl, alkenyl, alkynyl, cycloalkyl, aryl, arylalkyl, acyl, alkoxyacyl, aryloxyacyl, or aminoacyl groups;
R²is O, S, or NH;
A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; and
B is alkyl, arylalkyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; excluding the following compounds:
(i) a compound, wherein Y is a direct bond, Z is CO, R¹ is 1-(piperidin-4-yl)piperidin-4-yl, R² is O, A is phenyl, and B is methyl;
(ii) compounds, wherein Y is NH, Z is CO, R¹ is H, R² is O, A is phenyl, and B is N-substituted aminomethylphenyl, azabicyclooctyl, or azabicyclononyl;
(iii) compounds, wherein Y is NH, Z is CO, R² is O, A is phenyl, R¹ is alkyl, alkenyl, or alkynyl, and B is azabicyclooctyl, azabicyclononyl, or azabicyclodecyl; and
(iv) a compound, wherein Y is NMe, Z is CO, R² is O, R¹ is H, A is phenyl, and B is azabicyclononyl.

12. A pharmaceutical composition according to claim 11, wherein A and B of the compound of formula I are as defined in claim 2.

13. A pharmaceutical composition according to claim 11, wherein the compound is according to claim 9.

14. A pharmaceutical composition according to claim 11, wherein the compound is according to claim 10.

15. A method for regulating, modulating, or inhibiting angiogenesis, comprising administrating to a subject in need thereof a therapeutically effective amount of a compound of formula I or a pharmaceutically acceptable salt thereof; 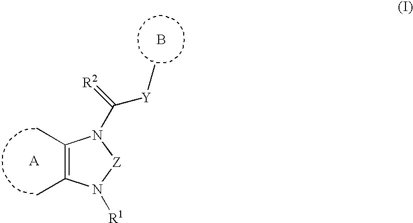
wherein:
Y is a direct bond or a linker group selected from a group of CH₂, NH, NR₁, S, SO, SO₂, or O;
Z is CO, CS, SO, SO₂, or C═NH;
R¹ is H, alkyl, alkenyl, alkynyl, cycloalkyl, aryl, arylalkyl, acyl, alkoxyacyl, aryloxyacyl, or aminoacyl groups;
R² is O, S, or NH;
A is one to three cycloalkyl or aryl ring groups, in which any of these ring groups may be connected with other ring through a single bond or fused with at least one other ring, and these ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11; and
B is alkyl, arylalkyl, or one to three cycloalkyl or aryl ring groups, in which any of ring groups may be connected with other ring through a single bond or fused with at least one other ring; and these alkyl, arylalkyl or ring groups are optionally substituted at one or more positions with alkyl, alkoxy, aryl, aryloxy, arylalkyl, arylalkyloxy, hydroxyl, halogen, trihalomethyl, S(O)R, SO₂NRR′, SO₃R, SR, B(OR)₂, PR₃, P(O)(OR)₂, OP(O)(OR)₂, NO₂, NRR′, OR, CN, C(O)R, NHC(O)R, (CH₂)ₙCO₂R, or CONRR′, wherein R and R′ are each independently selected from the group consisting of H, alkyl, alkoxy, aryl, aryloxy, arylalkyl, and arylalkyloxy, and n is 0-11.

16. The method of claim 15, wherein A and B of the compound of formula (I) are as defined in claim 2.

17. The method of claim 15, wherein the compound is according to claim 9.

18. The method of claim 15, wherein the compound is according to claim 10.

19. The method of claim 15, which is for preventive and/or therapeutic treatment of pathological state arising from unregulated angiogenesis.

20. The method of claim 19, wherein the pathological state is a proliferative, fibrotic or metabolic disease.

21. The method of claim 20, wherein the disease is selected from the group consisting of atherosclerosis, arthritis, cancer, diabetic retinopathy, fibrosis, psoriasis and restenosis.

22. The method of claim 15, which is used in conjunction with other treatments for treating or preventing pathological states arising from unregulated angiogenesis.
